(19) United States
(12) Patent Application Publication
(10) Pub. No.: US 2017/0231558 A1
(43) Pub. Date: Aug. 17, 2017

(54) Measurement Of ALS Progression Based On Kinetic Data

(71) Applicant: ALS Therapy Development Institute, Cambridge, MA (US)

(72) Inventors: Alan Gill, Reading, MA (US); Steven Perrin, Newbury, MA (US); Bashar Al-Nakhala, Hingham, MA (US)

(21) Appl. No.: 15/430,802

(22) Filed: Feb. 13, 2017

Related U.S. Application Data
(60) Provisional application No. 62/294,633, filed on Feb. 12, 2016.

(51) Int. Cl. A61B 5/00; A61B 5/11

(52) U.S. Cl. CPC A61B 5/4842; A61B 5/4082; A61B 5/11; A61B 5/6801; A61B 5/0015; A61B 2562/0219

(57) ABSTRACT

Methods and systems are disclosed for evaluating a neurological condition by employing at least one motion sensor, configured to be attached to a body appendage, a memory associated with the sensor(s) to periodically record movement data during periods of prescribed exercises; and a processor for analyzing changes in movement data over time to evaluate the progression of the neurological condition. In one embodiment, the neurological condition is ALS and at least four motion sensors are employed such that each arm and leg of the patient has an associated sensor. The sensors can be accelerometers that measure the displacement, velocity and acceleration of an associated limb during periods of prescribed exercise. For example, changes in the patient's ability to repeat a series of limb-lifting exercises or the measurement of limb tremors associated with the conduct of the exercises can be correlated with norms and analyzed to classify the stage of ALS in a patient and/or predict the rate of progression.

### RELATED APPLICATION

The present application claims priority to provisional application no. 62/294,633, filed Feb. 12, 2016, titled “Measurement Of ALS Progression Based On Kinetic Data.” This application incorporates the content of the provisional application by reference in its entirety.

### TECHNICAL FIELD

The present invention relates to the treatment of neurological disorders and, in particular, to the diagnosis and treatment of neurodegenerative diseases, such as Amyotrophic Lateral Sclerosis (ALS).

### BACKGROUND

ALS is a progressive neurological disorder characterized by muscle fiber atrophy resulting from the degeneration of motor neurons in the spinal column and brain. ALS affects approximately 30,000 U.S. citizens, with only ≈10% of cases being classified as the familial form of ALS. In a subset of familial patients with mutations in the metabolic enzyme superoxide dismutase 1 (SOD1), the pathological progression may be attributed to an unknown gain of function associated with a mutant form of the enzyme, i.e., is SOD1 dependent. However in the majority of ALS cases the SOD1 gene contains no mutations, the activity of the SOD1 enzyme is normal, and the mechanism of disease pathology is unknown, i.e., not SOD1 dependent. Therefore, the remaining 90% of ALS cases are classified as sporadic cases, with no well-characterized genetic component or causal agent.

Although ALS is characterized by loss of motor neurons in the spinal cord resulting in muscle atrophy, the disease also manifests itself with changes in axon transport, protein aggregation, excitotoxicity, astrocytosis, mitochondrial dysfunction, microglial activation and synaptic remodeling. Microglial activation, astrocytosis and the presence of infiltrating inflammatory cells from outside the central nervous system have been well described. There is accumulation of IgG immunoreactive deposits in the spinal cord of ALS patients, infiltration of lymphocytes, dendritic cells, monocytes, and macrophages into the spinal cord in ALS.

Although at present ALS is inevitably progressive, there is considerable variation in the rate of progression and the length of life expectancy. Currently, more than half of those diagnosed with ALS will survive three or more years, 20 percent live at least 5 years and as many as ten percent live for 10 years or more.

Determining the stage of ALS in an individual patient can be difficult and, to some extent, subjective. One predominant measure of ALS progression relies upon self-assessment using the ALS Functional Rating Scale (ALSFRS) questionnaire, in which higher numbers represent better physiological condition. The ALSFRS form consists of 12 questions each scored from 0 to 4, addressing the ability of the patient to perform certain actions, such as upper limb function, lower limb function, swallowing and speaking functions, the use of feeding tubes, and the need for respiration assistance.

There exists a need for methods and systems that can evaluate ALS in patients in a more quantitative manner than a self-assessment questionnaire.

### SUMMARY

Methods and systems are disclosed for evaluating a neurological condition by employing at least one, and preferably multiple, motion sensors, each configured to be attached to a different body appendage, a memory associated with the sensors to periodically record movement data during periods of prescribed exercises; and a processor for analyzing changes in movement data over time to evaluate the progression of the neurological condition. In one embodiment, the neurological condition is ALS and at least four motion sensors are employed such that each arm and leg of the patient has an associated sensor. The sensors can be accelerometers that measure the displacement, velocity and acceleration of an associated limb during periods of prescribed exercise. For example, changes in the patient's ability to repeat a series of limb-lifting exercises or the measurement of limb tremors associated with the conduct of the exercises can be correlated with a baseline and/or normalized values and analyzed to classify the stage of ALS in a patient and/or predict the rate of progression.

In one aspect of the invention, systems for evaluating the progression of ALS are disclosed that can include at least a first accelerometer attachable to a first body limb of a patient and configured to measure first movement data, including acceleration of the first limb during a period of prescribed exercise, and a processor for analyzing the movement data over time to evaluate the progression of ALS in the patient.

The systems can further include a second accelerometer attachable to a second body limb of the patient and configured to measure second movement data, including the acceleration of the second limb during the period of prescribed exercise. Moreover, the system can include a third accelerometer attachable to a third body limb and configured to measure third movement data, including the acceleration of the third limb during the period of prescribed exercise. The system can further include a fourth accelerometer attachable to a fourth body limb performing these tasks. The body limbs are selected from the group consisting of a left arm, a right arm, a left leg and a right leg. For example, an arm (left or right) consists of any region between a shoulder and a hand. This includes an arm, a forearm, and a wrist. A leg consists of any region between a hip and a foot. This includes an upper leg, a lower leg and an ankle. In one preferred embodiment, four accelerometers are deployed on each of the wrists and ankles of the patient to obtain data from all four limbs.

In certain embodiments the system's processor analyzes the movement data by comparing one or more acceleration vector magnitudes (VM) over time, the acceleration vector magnitudes being defined by an equation:

VM=\sqrt{x²+y²+z²}

wherein x, y, and z represent the magnitudes of limb acceleration as measured in the x, y and z directions.

The accelerometer can be integrated into a sensor unit that includes a power supply, e.g., batteries, and a transmitter able to communicate wirelessly, e.g., by FM, RF or Bluetooth formats, with the processor (or a base station that relays the data to a remote processor) by wired connection, via the interne, by telephony, by cellular communications or any other suitable data transmission medium. The sensor can also include a memory that stores various reading and uploads data (or multiple data sets) to the processor in real time, at a later time or according to a predefined schedule.

In another aspect of the invention, methods of evaluating the progression of ALS in a patient are disclosed that can include the following steps of attaching at least a first accelerometer to a first body limb of a patient, detecting movement data, including the acceleration of the one or more limbs during a period of prescribed exercise, optionally storing the movement data in a memory associated with one or more of the accelerometers (or a base station in communication therewith), and outputting the movement data from the accelerometer or the memory to a processor for analyzing the movement data over time to evaluate the progression of ALS in the patient.

The methods can further include attaching a second accelerometer to a second body limb of the patient, attaching a third accelerometer to a third body limb of the patient or attaching accelerometers to all four limbs and outputting the data from the respective accelerometers to the processor to analyze each of the limb functions measured For example, the body limbs are selected from the group consisting of a left arm, a right arm, a left leg and a right leg, and in one preferred embodiment, four accelerometers are deployed on each of the wrists and ankles of the patient to obtain data from all four limbs.

In certain embodiments the methods can include analyzing the movement data by comparing acceleration vector magnitudes (VM) over time, the acceleration vector magnitudes being defined by the afore-described equation, wherein x, y, and z represent the magnitudes of limb acceleration as measured in the x, y and z directions. A decline in the average vector magnitude in one or more body limbs can indicate progression of ALS in the patient.

The methods of the present invention can further include the step of providing therapy or palliative care based on the measured score or analysis of patient function.

### BRIEF DESCRIPTION OF THE DRAWINGS

FIG. 1 is a graph of acceleration vector magnitude measurements from an illustrative set of subjects, showing the range of responses during a prescribed exercise period.

FIG. 2 is a graph of acceleration vector magnitude measurements from a single subject, at each limb, showing the range of responses during a prescribed exercise period.

FIG. 3 is a graph of acceleration vector magnitude measurements from a different subject, at each limb, showing the range of responses during a prescribed exercise period.

FIG. 4 is a plot of average acceleration vector magnitude measurements from all subjects at each limb, during the study period.

FIG. 5 is a plot of average acceleration vector magnitude measurement from a single subject at each limb, during the study period.

FIG. 6 is a plot of average acceleration vector magnitude measurement from a different subject at each limb, during the study period.

FIG. 7 is a plot of the self-reported ALSFRS score from the subject of FIG. 6, during the study period.

FIG. 8 is a plot of average acceleration vector magnitude measurement from another subject at each limb, during the study period.

FIG. 9 is a plot of average acceleration vector magnitude measurement from another subject at each limb, during the study period.

FIG. 10 is a plot of average acceleration vector magnitude measurement from another subject at each limb, during the study period.

FIG. 11 is a plot of average acceleration vector magnitude measurement from another subject at each limb, during the study period.

### DETAILED DESCRIPTION

The present invention generally relates to devices, systems and methods for evaluating a neurological condition by employing a plurality of motion sensors. Each device, i.e., motion sensor, can be configured to be attached to a different body appendage. A memory can be associated with the plurality of sensors to periodically record movement data during periods of prescribed exercises. The devices and systems described herein can also include a processor for analyzing changes in movement data over time to evaluate the progression of the neurological condition. In one embodiment, the neurological condition is ALS and at least two motion sensors are employed such that one or both arms, one or both legs, or a combination thereof, of the patient has an associated sensor. The sensors can be accelerometers that measure the displacement, velocity and acceleration of an associated limb during periods of prescribed exercise. For example, changes in the patient's ability to repeat a series of limb-lifting exercises or the measurement of limb tremors associated with the conduct of the exercises can be correlated with norms and analyzed to classify the stage of ALS in a patient and/or predict the rate of progression.

The term “accelerometer” as used herein is intended to encompass instruments capable of measuring acceleration (changes in velocity) in at least one direction relative to an inertial frame. In certain embodiments, multiaxial accelerometer can used , for example, to measure changes in velocity relative to a three-dimensional Cartesian coordinate system (x, y and z directions). In some applications, two-axis or even one-axis measurements may suffice. Alternatively, acceleration can be measured based on non-Cartesian, e.g., cylindrical or spherical coordinates.

As used herein, the term “limb” or “appendage” refers to an arm and/or a leg. A body limb is selected from the group consisting of a left arm, a right arm, a left leg and a right leg. For example, an arm (left or right) consists of any region between a shoulder and a hand. This includes an arm, a forearm, and a wrist. A leg consists of any region between a hip and a foot. This includes an upper leg, a lower leg and an ankle.

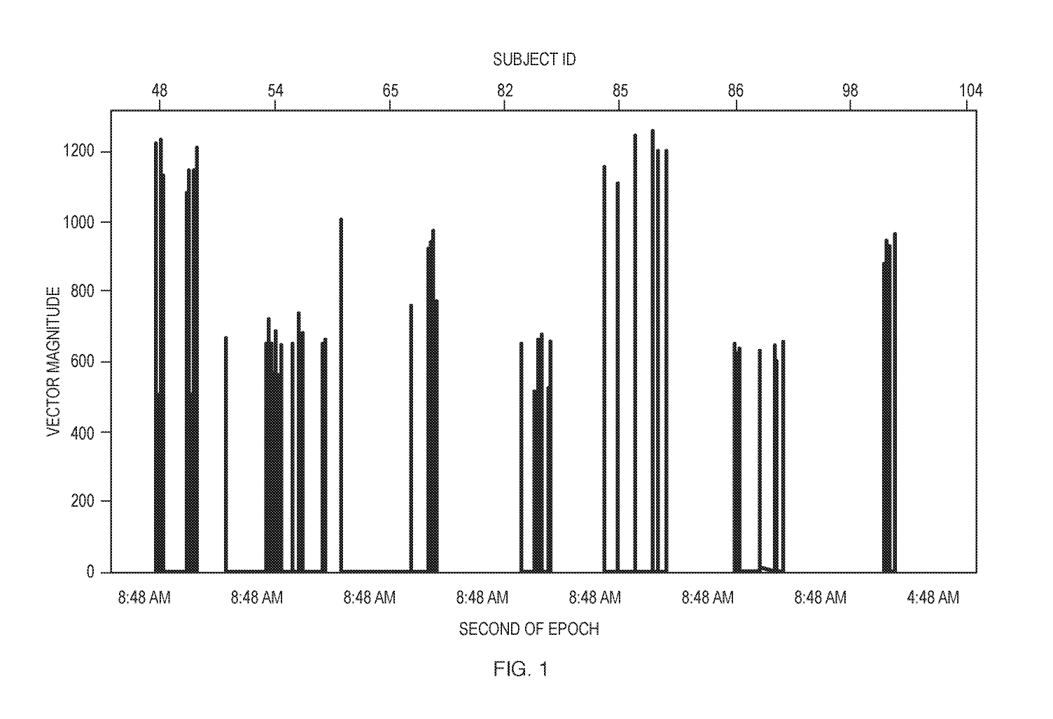

In FIG. 1 is a graph is shown of acceleration vector magnitude measurements from an illustrative set of subjects, showing the range of responses during a prescribed exercise period. Each bar represents the composite vector magnitude for an individual patient.

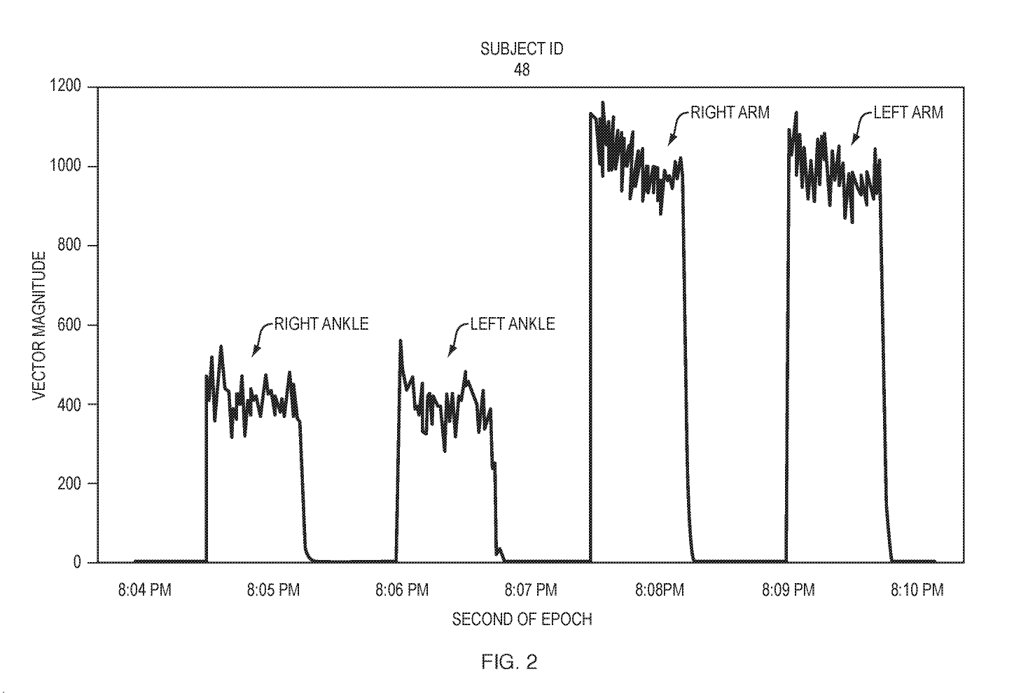

FIG. 2 is a graph of acceleration vector magnitude measurements from a single subject, at each limb, showing the range of responses during a prescribed exercise period. The prescribed exercise required the patient to raise and lower their arms and legs ten times for each limb.

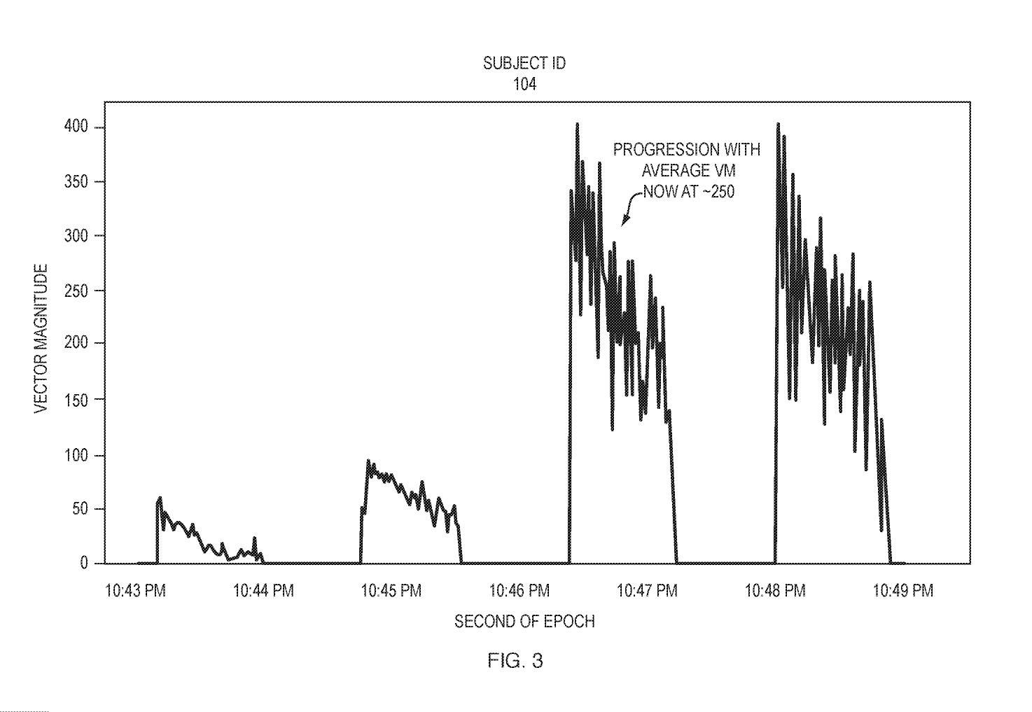

FIG. 3 is a graph of acceleration vector magnitude measurements from a different subject, at each limb, showing the range of responses during a prescribed exercise period. As can be observed, the patient had difficulty completing the exercise regime.

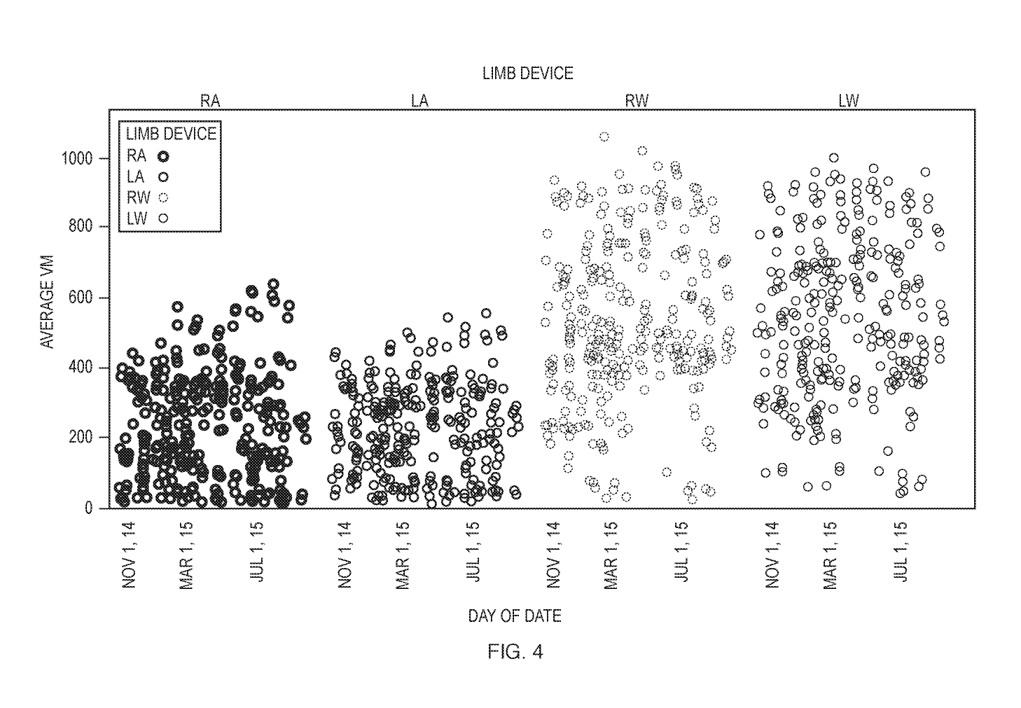

FIG. 4 is a plot of average acceleration vector magnitude measurements from all subjects at each limb, over a period spanning eight months. (RA signifies right ankle, LA signifies left ankle, RW signifies right wrist and LW signifies left wrist.) The prescribed exercise program called for three repetitions of the same exercise on each test day.

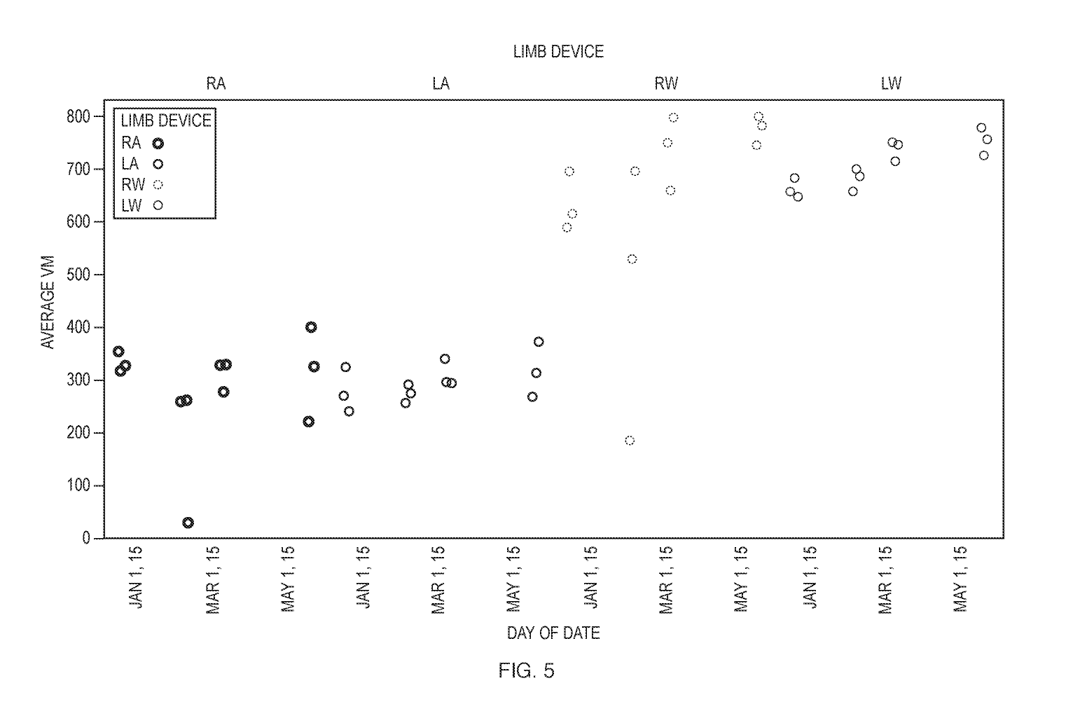

FIG. 5 is a plot of average acceleration vector magnitude measurement from a single subject at each limb, during a five-month period. As can be seen more clearly in this graph, the test consisted of three repetitions of the same exercise program on each of four test days during the five-month study. There is little to no change in this patient's score over the test period, suggesting that the patient has a slow form of ALS progression.

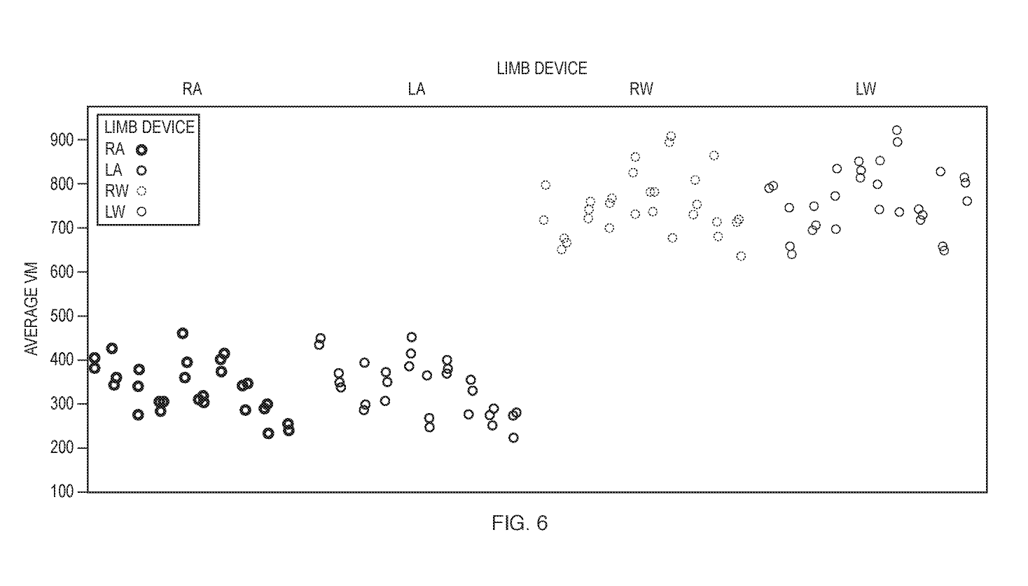

FIG. 6 is a plot of average acceleration vector magnitude measurement from a different subject at each limb, during an eight-month study period. Again, the test consisted of three repetitions of the same exercise program on each of the test days during the study. There is little to no change in this patient's upper body scores over the test period. However, slow progressive diminution can be seen in this patient's strength in both the left and right legs.

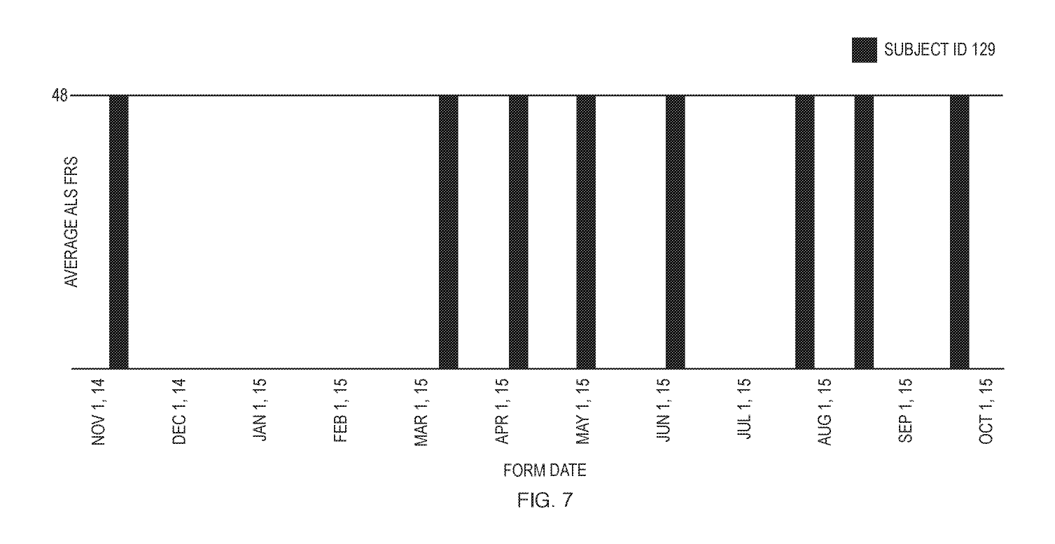

FIG. 7 is a plot of the self-reported ALSFRS scores from the subject of FIG. 7, during a one-year period that overlapped with the accelerometer study period. This patient self-reported a perfect score of 48 for the entire period spanning the accelerometer test study.

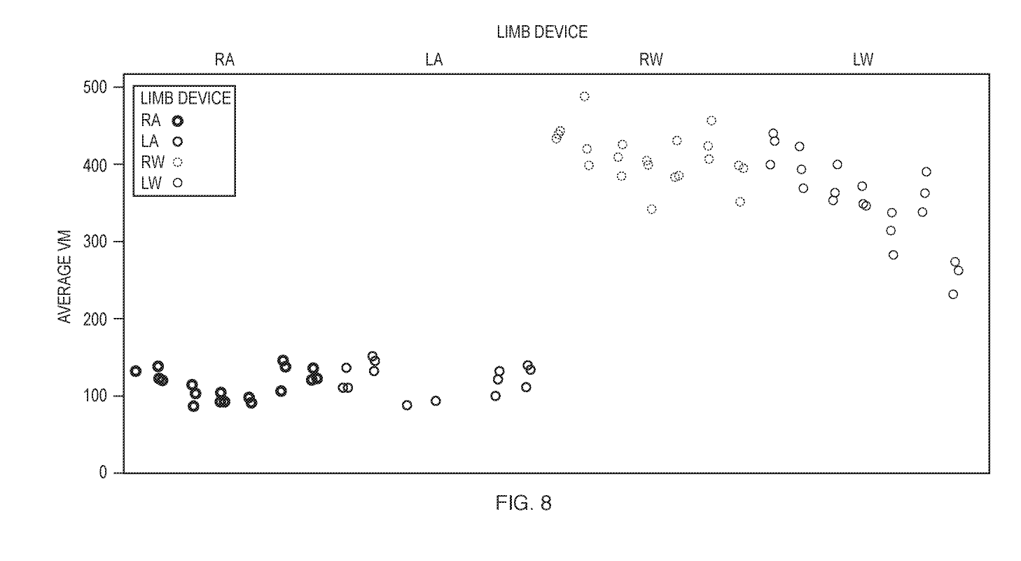

FIG. 8 is a plot of average acceleration vector magnitude measurement from another subject at each limb, during a six-month study period. Again, the test consisted of three repetitions of the same exercise program on each of the test days during the study. There is little to no change in this patient's lower body scores over the test period. However, moderate progression of ALS can be seen in this patient's strength in both the left and right arms.

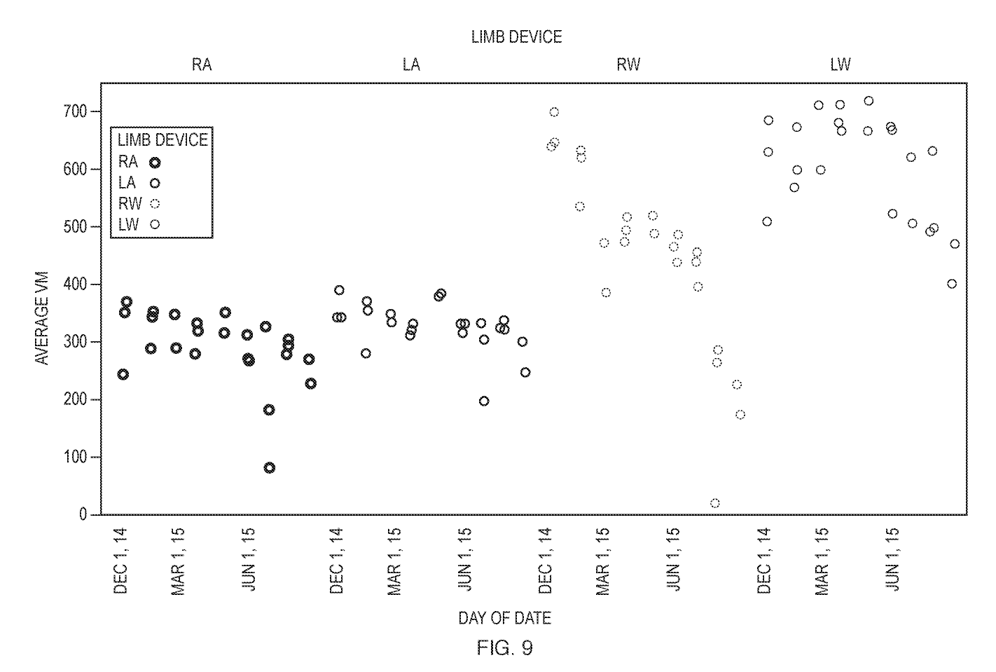

FIG. 9 is a plot of average acceleration vector magnitude measurement from another subject at each limb, during a six-month study period. Again, the test consisted of three repetitions of the same exercise program on each of the test days during the study. There is little to no change in this patient's lower body scores over the test period. However, faster progression of ALS can be seen in this patient's strength in both the left and right arms.

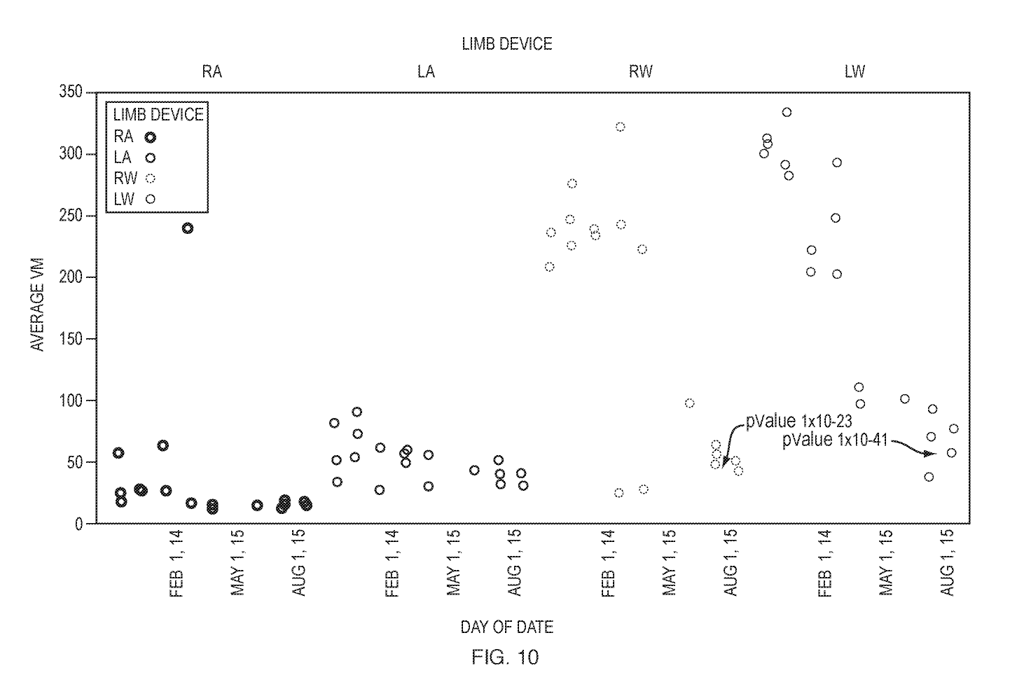

FIG. 10 is a plot of average acceleration vector magnitude measurement from another subject at each limb, during an eight-month period. The test consisted of two or three repetitions of the same exercise program on each of the test days during the study if possible for the patient. The lower body scores suggest that substantial diminution in the patients lower body strength had already occurred prior to the study period. Moreover, significant progression of ALS can be seen in this patient's strength in both the left and right arms during the study period.

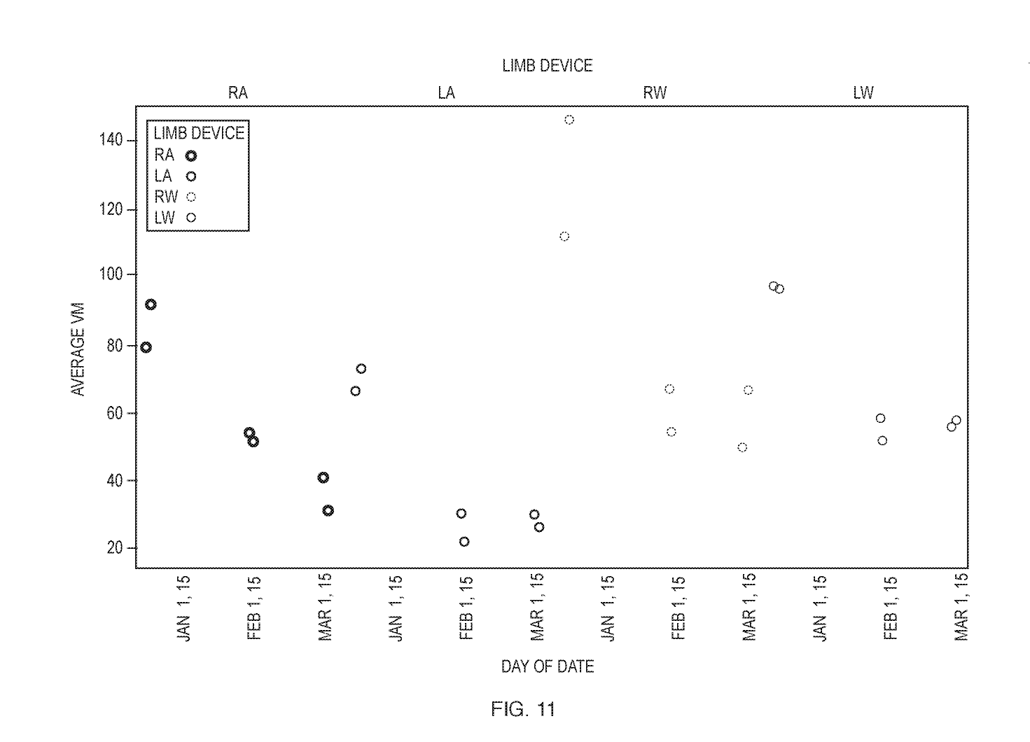

FIG. 11 is a plot of average acceleration vector magnitude measurement from another subject at each limb, during a three-month study period. The test consisted of two repetitions of the same exercise program on each of the test days during the study. The patient exhibited fast declines in scores for all four limbs.

The studies demonstrate that kinetic data, especially acceleration vector magnitude measures can be used to assess the stage and rate of progression of neurological diseases, especially in ALS.

1. A system for evaluating the progression of ALS comprising:
a first accelerometer attachable to a first body limb of a patient and configured to measure first movement data, including acceleration of the first limb during a period of prescribed exercise;
a second accelerometer attachable to a second body limb of the patient and configured to measure second movement data, including the acceleration of the second limb during the period of prescribed exercise;
a memory associated with the accelerometers to record limb movement data; and
a processor for analyzing the movement data over time to evaluate the progression of ALS in the patient.

2. The system of claim 1, wherein the processor analyzes the movement data by comparing acceleration vector magnitudes (VM) over time, the acceleration vector magnitudes being defined by an equation:
VM=\sqrt{x²+y²+z²}
wherein x, y, and z represent the magnitudes of limb acceleration as measured in the x, y and z directions.

3. The system of claim 1, further comprising a third accelerometer attachable to a third body limb and configured to measure third movement data, including the acceleration of the third limb during the period of prescribed exercise.

4. The system of claim 3, further comprising a fourth accelerometer attachable to a fourth body limb and configured to measure fourth movement data, including the acceleration of the fourth limb during the period of prescribed exercise.

5. The system of claim 2, wherein the first body limb and the second body limb are selected from the group consisting of a left arm, a right arm, a left leg and a right leg.

6. The system of claim 2, wherein the period of prescribed exercise comprises a leg exercise, an arm exercise, or a combination thereof.

7. The system of claim 6, wherein the leg exercise, the arm exercise, or the combination thereof is performed while the patient is in a sitting position.

8. The system of claim 1, wherein the system further comprise a wireless transmitter for communicating movement data to the processor.

9. A method for evaluating the progression of ALS in a patient comprising:
attaching a first accelerometer to a first body limb of a patient,
attaching a second accelerometer to a second body limb of the patient,
detecting movement, including the movement of the first and second limbs during a period of prescribed exercise,
storing movement data in a memory associated with the accelerometers, and
outputting the movement data from the memory to a processor for analyzing the movement data over time to evaluate the progression of ALS in the patient.

10. The method of claim 9, wherein the processor analyzes the movement data by comparing acceleration vector magnitudes (VM) over time, the acceleration vector magnitudes being defined by an equation:
VM=\sqrt{x²+y²+z²}
wherein x, y, and z represent the magnitudes of limb acceleration as measured in the x, y and z directions.

11. The method of claim 9, wherein the first body limb and the second body limb are selected from the group consisting of a left arm, a right arm, a left leg and a right leg.

12. The method of claim 9 wherein the method further comprises detecting movement data over time by sequentially measuring movement data during prescribed exercises at a plurality of different times.

13. The method of claim 9, further comprising attaching a third accelerometer to a third body limb of the patient.

14. The method of claim 13, further comprising attaching a fourth accelerometer to a fourth body limb of the patient.

15. The method of claim 14, further comprising detecting movement data, including the acceleration of the third and fourth limbs during the period of prescribed exercise.

16. The method of claim 15, wherein a decline in the average vector magnitude in one or more body limbs indicates progression of ALS in the patient.
